(19) United States
(12) Patent Application Publication
(10) Pub. No.: US 2024/0138714 A1
(43) Pub. Date: May 2, 2024

(54) COMMUNICATION METHOD AND SYSTEM THROUGH HUMAN BODY

(71) Applicant: Luis Stohr, Redondo Beach, CA (US)

(72) Inventor: Luis Stohr, Redondo Beach, CA (US)

(21) Appl. No.: 18/198,211

(22) Filed: May 16, 2023

Related U.S. Application Data
(63) Continuation-in-part of application No. 17/975,262, filed on Oct. 27, 2022, now Pat. No. 11,683,633.

(51) Int. Cl. A61B 5/11; A61B 5/00

(52) U.S. Cl. CPC A61B 5/1126; A61B 5/682; A61B 5/6826; A61B 2562/0219

(57) ABSTRACT

A system for communicating through a human body includes a wearable device including an accelerometer configured to receive motion signals in response to motions of the human body and a first decoder configured to transform motion signals into electrical signals; a memory configured to store one or more instructions; and a processor communicated with the wearable device and configured to execute the instructions to: determine a pattern of the motions of the human body from the electrical signals; and compare the pattern of the motions of the human body with a predetermined pattern of motions of the human body to determine whether the pattern of the motions of the human body matches with the predetermined pattern of motions of human body.

### FIELD OF THE DISCLOSURE

The present disclosure relates to a communication method and system for efficiently generating responses consisting of answering questions most simply, with the quickest and lowest human body energy possible: the tong and/or a finger.

### BACKGROUND OF THE DISCLOSURE

Generally, people need to respond to questions efficiently, quickly, undistracted, and with the lowest body energy consumption. In today's daily life, within a society based on an increased amount of communication created by the Internet, multitasking is helpful, and quick-short answers with precise information without distracting duties are needed for most people.

Waving the head up and down or sideways for yes or no, or the “Air Writing” method where people write the letter in the air, even doing symbolic finger movements such as “thumb up” for OK on texting or “crossing fingers” for WISHING are communication approaches to simplify answering questions in any language or culture, without languages boundaries. Therefore, only a finger or the tongue moves to respond to questions is convenient for certain situations.

Currently, the internet and mobile devices reduce communication questions and special answers by using gestures or simply waving the head or doing a facial expression to respond to questions. Also, people are unreachable or busy with other activities and do not want to engage in two-way communication with another person looking for an answer, so efficient access to short answers during communication is highly needed without distraction.

All referenced patents, applications, and literature are incorporated herein by reference. Furthermore, where a definition or use of a term in a reference, incorporated by reference herein, is inconsistent or contrary to the definition of that term provided herein, the definition provided herein applies, and the definition of that term in the reference does not apply. The disclosed embodiments may seek to satisfy one or more of the above-mentioned desires. Although the present embodiments may obviate one or more of the above-mentioned desires, it should be understood that some aspects of the embodiments might not necessarily obviate them.

### BRIEF SUMMARY OF THE DISCLOSURE

In a general implementation, a system for communicating through a human body may comprise a wearable device comprising an accelerometer configured to receive motion signals in response to motions of the human body and a first decoder configured to transform motion signals to electrical signals; a memory configured to store one or more instructions; and a processor communicated with the wearable device and configured to execute the instructions to: determine a pattern of motions of the human body from the electrical signals; and compare the pattern of the motions of the human body with a predetermined pattern of motions of the human body to determine whether the pattern of the motions of the human body matches with the predetermined patterns of motions of the human body.

In another aspect combinable with the general implementation, at least one of the wearable devices is worn on the fingers of the human body to detect motions of the human fingers.

Among the many possible implementations of the system, wherein the processor executes the instructions to:
if the pattern of the motions of the human body matches with the predetermined pattern of motions of the human body, generating responding data based on the predetermined pattern of motions of the human body.

Further, it is contemplated that the instructions for determining the pattern of the motions of the human body from the electrical signals comprise instructions for the system to:
store the predetermined pattern of the motions of the human body in the memory.

In the alternative, the predetermined pattern of motions of the human body comprises a direction, a frequency, and a dwell time of the motions of the human body.

It is still further contemplated that the responding data comprises readable word information, audio information, and vibration information.

In another aspect combinable with the general implementation, the system may further comprise a tongue sensor device placed inside a mouth of the human body and communicated with the processor, wherein the tongue sensor device comprises a plurality of switch sensors to receive first motion signals in response to motions of the human tongue.

In another aspect combinable with the general implementation, the tongue sensor device comprises a second decoder configured to transform first motion signals to first electrical signals.

In another aspect combinable with the general implementation, the processor executes the instructions to: determine a pattern of the motions of the human tongue from the first electrical signals; and compare the pattern of the motions of the human tongue with a predetermined pattern of motions of the human tongue to determine whether the pattern of the motions of the human tongue matches with the predetermined pattern of the motions of the human tongue.

In another aspect combinable with the general implementation, the processor executes the instructions to: if the pattern of the motions of the human tongue matches with the predetermined pattern of motions of the human tongue, generating first responding data based on the predetermined pattern of motions of the human tongue.

In another aspect combinable with the general implementation, the instructions for determining the pattern of the motions of the human tongue from the first electrical signals comprises instructions for the system to:
store the predetermined pattern of motions of the human tongue in the memory.

In another aspect combinable with the general implementation, the first responding data comprises readable word information, audio information, and/or vibration information.

Another aspect of the embodiment is directed to a communication method through a human body by a communication system, comprising:
receiving, by an accelerometer of a wearable device, motion signals in response to motions of the human body;
transforming, by a first decoder of a wearable device, motion signals to electrical signals;
storing, by the memory, one or more instructions;
determining, by the processor, a pattern of the motion of the human body from the electrical signals; and
comparing, by the processor, the pattern of the motions of the human body with a predetermined pattern of motions of the human body to determine whether the patterns of the motions of the human fingers match with the predetermined patterns of motions of the human body.

Accordingly, the wearable device is worn on the fingers of the human body to detect the motions of the human fingers.

In some embodiments, the step of comparing, by the processor, the pattern of the motions of the human body with a predetermined pattern of motions of the human body to determine whether the patterns of the motions of the human body match with the predetermined patterns of motions of human body further comprises:
if the pattern of the motions of the human body matches with the predetermined pattern of motions of the human body, generate responding data based on the predetermined pattern of motions of the human body.

In another aspect combinable with the general implementation, the step of determining, by the processor, the pattern of the motion of the human body from the electrical signals further comprises: storing the predetermined pattern of motions of the human body in the memory.

In another aspect combinable with the general implementation, the method may further comprise steps of:
placing a tongue sensor device placed inside a mouth of the human body;
receiving, by a plurality of switch sensors of the tongue sensor device, first motion signal in response to motions of human tongue; and
transforming, by a second decoder of the tongue sensor device, the first motion signals to the first electrical signals.

In the alternative, the method further comprises steps of:
determining, by the processor, a pattern of the motions of the human tongue from the first electrical signals; and
comparing, by the processor, the pattern of the motions of the human tongue with a predetermined pattern of motions of the human tongue to determine whether the pattern of the motions of the human tongue matches the predetermined pattern of the motions of the human tongue.

In another aspect combinable with the general implementation, the step of comparing the pattern of the motions of the human tongue with a predetermined pattern of the motions of the human tongue to determine whether the pattern of the motions of the human tongue matches the predetermined pattern of the motions of the human tongue further comprises:
if the pattern of the motions of the human tongue matches with the predetermined pattern of the motions of the human tongue, generating first responding data, including readable word information, audio information, and/or vibration information, based on the predetermined pattern of the motions of the human tongue.

Further, it is contemplated that the step of determining, by the processor, the pattern of the motions of the human tongue from the first electrical signals further comprises steps of:
storing the predetermined pattern of the motions of the human tongue in the memory. While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features described in the context of a single implementation can also be implemented in multiple implementations separately or in any suitable subcombination.

Moreover, although features may be described above and below as acting in certain combinations and even initially claimed as such, one or more features from a claimed combination can in some cases be excised from the combination, and the claimed combination may be directed to a subcombination or variation of a subcombination.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

It should be noted that the drawing figures may be in simplified form and might not be too precise scale. In reference to the disclosure herein, for purposes of convenience and clarity, only directional terms such as top, bottom, left, right, up, down, over, above, below, beneath, rear, front, distal, and proximal are used with respect to the accompanying drawings. Such directional terms should not be construed to limit the scope of the embodiment in any manner.

FIG. 1 is a schematic view of a communication system through the human body according to an aspect of the embodiment.

FIG. 2 is a schematic view of a communication system through the human body according to an aspect of the embodiment.

FIG. 3 is a diagram view of a communication system through the human body according to an aspect of the embodiment.

FIG. 4 is a diagrammatic view of a communication system through the human body according to an aspect of the embodiment.

FIG. 5 shows the pattern and the predetermined pattern of motions of the human body according to an aspect of the embodiment.

FIG. 6 is a diagrammatic view of a communication system through the human body according to an aspect of the embodiment.

FIGS. 7-10 are diagram views of a communication method through the human body according to an aspect of the embodiment.

FIG. 11 shows the experiment results according to an aspect of the embodiment.

### DETAILED DESCRIPTION OF THE EMBODIMENTS

The different aspects of the various embodiments can now be better understood by turning to the following detailed description of the embodiments, which are presented as illustrated examples of the embodiments defined in the claims. It is expressly understood that the embodiments as defined by the claims may be broader than the illustrated embodiments described below.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

It shall be understood that the term “means,” as used herein, shall be given its broadest possible interpretation in accordance with 35 U.S.C., Section 112(f). Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

Unless defined otherwise, all technical and position terms used herein have the same meaning as commonly understood by one of ordinary skill in the art to which the invention pertains. Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

As used herein, “a wearable device” and “a tongue sensor device” refer to a “Human Unilateral Harangue” or “HUH” in the present invention.

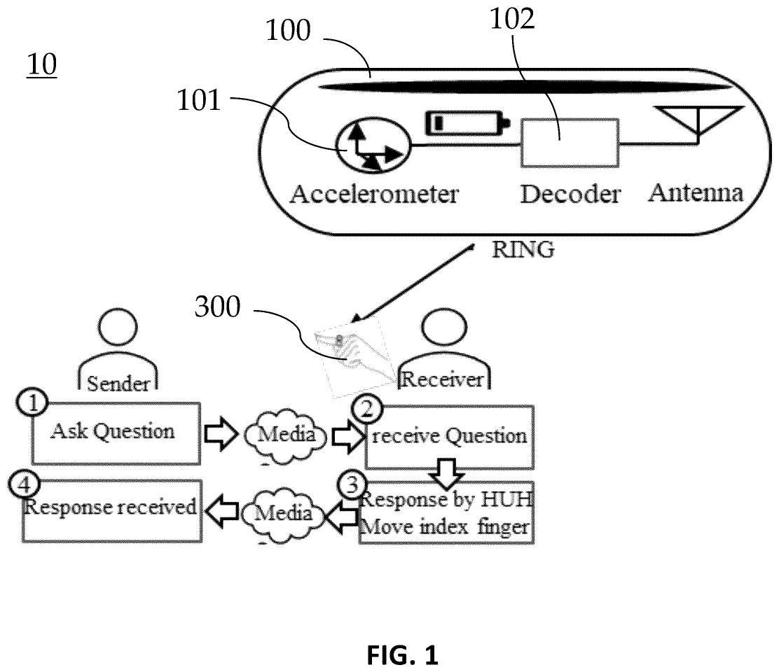

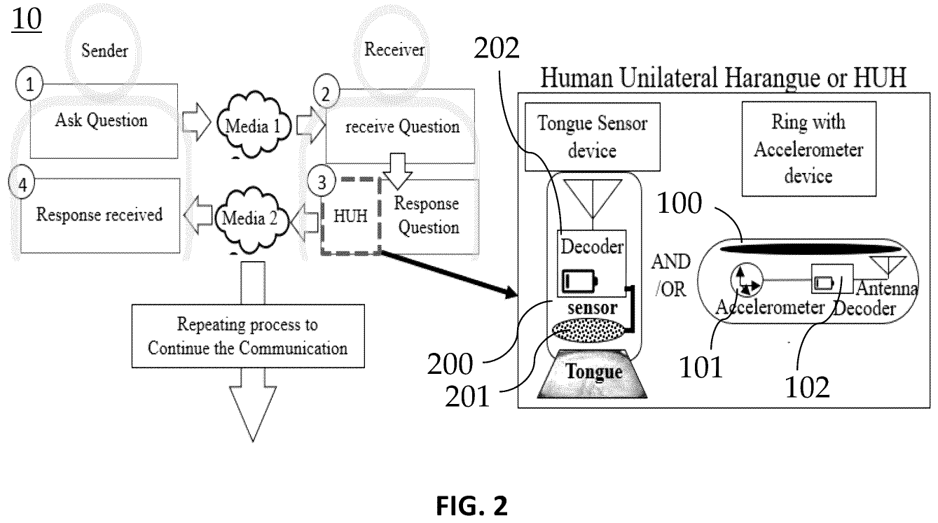

FIGS. 1-2 generally depict a communication system 10 through a human body according to an aspect of the embodiment.

Referring to FIG. 1, the system 10 may comprise a wearable device 100 adapted to be worn on a human body (preferably human fingers) 300, wherein the wearable device 100 may comprise an accelerometer 101 configured to receive motion signals in response to motions of fingers of the human body and a first decoder 102 configured to transform motion signals to electrical signals.

Continuing to FIG. 2, the system 10 may further comprise a tongue sensor device 200 placed inside a mouth of the human body, wherein the tongue sensor device 200 may comprise a plurality of switch sensors 201 to receive first motion signals in response to the motions of the human tongue. In some embodiments, the tongue sensor device 200 may comprise a second decoder 202 configured to transform first motion signals to first electrical signals.

In some embodiments, the wearable device 100 and/or the tongue sensor device 200 (“Human Unilateral Harangue” or “HUH”) may capture the simple response from a receiver by detecting motions of the finger or tongue of the human body, such as motion signals, generated from a sender, wherein the motion signals may be transformed to electrical signals by the first decoder 102. In some embodiments, the motion signals may be transmitted to the wearable device 100 via media. In some embodiments, the motion signals may comprise movements/motions from the finger or fingers and the tongue of the human body, and in order words, it is not only fingers or the tongue that can be used by the receiver to respond to the question, but also any part of the human body can be included on this invention. In some embodiments, the accelerometer 101 may also comprise tactile, movement, sound visual or vibration sensors or sensors, tactile sensors, mercury switches, gravity switches, visual sensors, switches activated by kinetics, and/or any type of chemical sensor or sensors.

In some embodiments, the wearable device 100 and/or the tongue sensor device 200 may capture the simple response from the receiver by detecting the movements/motions of the finger or tongue. When the sender asks a question, the inquiring signals may be transported through the media and perceived by the receiver. The receiver resonates and responds to a simple answer using a tongue or a finger/s movement/motion, which is captured by the wearable device 100 and/or the tongue sensor device 200 (HUH apparatus) detecting the movements/motions of the tongue or finger/s (or other ways of human response). The motion signals may be decoded into electrical signals by the first decoder, 102, and/or the second decoder 202. While the receiver receives the question generated from the sender, the receiver may generate responses through the wearable device 100 and/or the tongue sensor device 200. Completing the communication cycle and repeating the question-answer are required. The motion signals generated by the received may be sent back to the sender, called “Response Received” in FIG. 2. This present invention also includes “other ways of human response,” which are and not only includes how a person responds to a question through voice, visual, or any other human corporal part used for communication by not only humans but animals too.

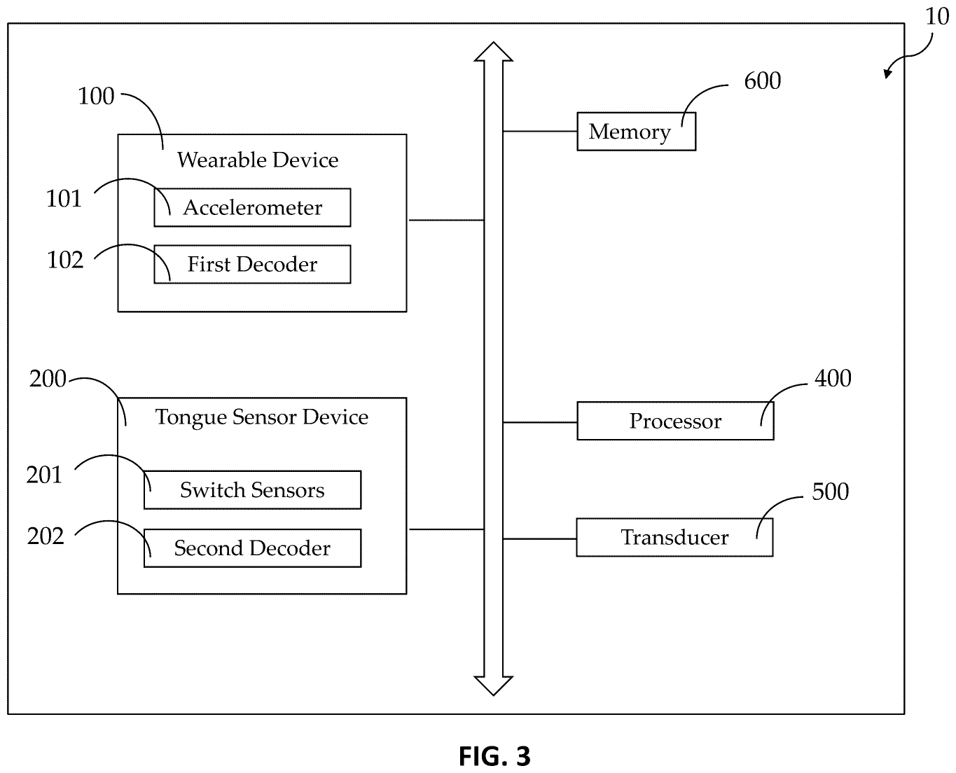

FIG. 3 generally depicts the communication system 10 through the human body according to an embodiment aspect.

Referring to FIG. 3, the system 10 may further comprise a memory 600 configured to store one or more instructions and a processor 400 communicating with the wearable device 100 and/or the tongue sensor device 200, wherein the processor 400 executes the instructions to:
determine a pattern of the motions of the human body (human fingers) from the electrical signals; and
compare the pattern of the motions of the human body (human fingers) with a predetermined pattern of motions of the human body (human fingers) to determine whether the pattern of the motions of the human body (human fingers) matches the predetermined pattern of motions of the human body (human fingers).

In some embodiments, the processor 400 executes the instructions to: if the pattern of the motions of the human body (human fingers) matches with the predetermined pattern of motions of the human body (human fingers), generating responding data based on the predetermined pattern of motions of the human body (human fingers).

In some embodiments, the instructions for determining the pattern of the motions of the human body (human fingers) from the electrical signals comprise instructions for the system to:
store the predetermined pattern of the motions of the human body (human fingers) in the memory.

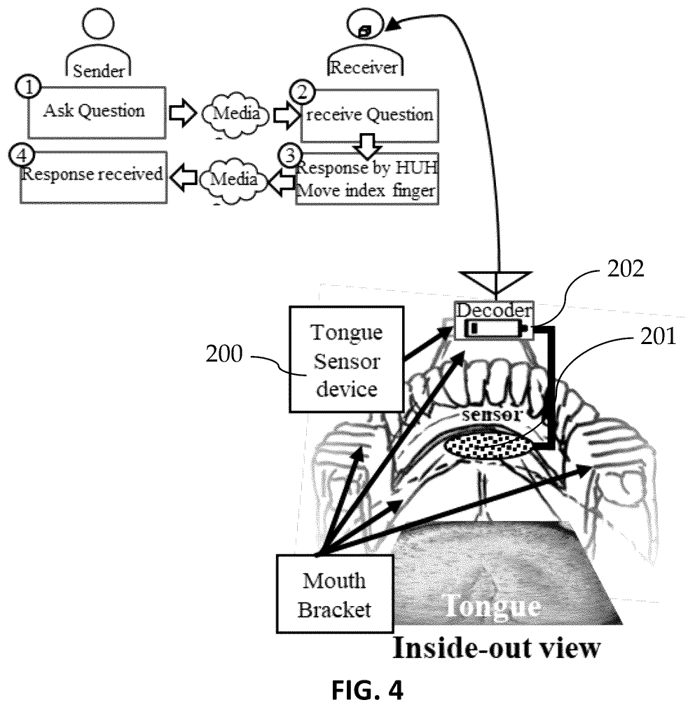

FIG. 4 generally depicts the tongue sensor device 200 placed inside the mouth of the human body for communication according to an aspect of the embodiment.

Referring to FIG. 4, the tongue sensor device 200 may cooperate with a mouth bracket, wherein the mouth bracket may be coupled to the teeth inside the human mouth. In some embodiments, the tongue sensor device 200 may be coupled with the mouth bracket; in such a way, the tongue sensor device 200 may be affixed on/adjacent to the teeth inside the human mouth.

In some embodiment, the tongue sensor device 200 may communicate with the processor 400, wherein the processor 400 may execute the instructions to: determine a pattern of the motions of the human tongue from the first electrical signals; and compare the pattern of the motions of the human tongue with a predetermined pattern of motions of the human tongue to determine whether the pattern of the motions of the human tongue matches with the predetermined pattern of motions of the human tongue.

In some embodiments, referring back to FIG. 3, the processor 400 executes the instructions to: if the pattern of the motions of the human tongue matches with the predetermined pattern of motions of the human tongue, generating first responding data based on the predetermined pattern of motions of the human tongue.

In some embodiments, the instructions for determining the pattern of the motions of the human tongue from the first electrical signals may further comprise instructions for the system to:
store the predetermined pattern of the motions of the human tongue in the memory 600.

Accordingly, in some embodiments, as shown in further detail in FIGS. 1 and 4, the sender may initiate a communication (ask a question/message). In such a way, the question may be transferred to the wearable device 100 and/or the tongue sensor device 200 through a media, and in such a manner, the message may be sent to the receiver. The receiver may decode or interpret the message, to generate responses based on the message. In some embodiments, the receiver may utilize the wearable device 100 and/or the tongue sensor device 200 to generate the corresponding responses. In other words, the receiver can generate the corresponding responses even when they are unavailable.

In some embodiments, the media may include any public messaging media and electronic communication made possible by computer and digital technologies. In some embodiments, the media may be the Internet, the world wide web, digital video cameras, cellular telephones, and cable and satellite television and radio. They enable quick, interactive, targeted, and potentially democratic communication, such as social media, blogs, podcasts, websites, wikis, instant messaging, and e-mail.

In some embodiments, the media may comprise: verbal, non-verbal, written, listening, and visual transmission. The media is sent by the sender and responded to by the receiver through any of the pointed communication media. In some embodiments, the media may include traditional social networking sites, such as social networking sites like Facebook, Twitter, LinkedIn, and TikTok, social review sites, image and video sharing sites, video hosting sites, community blogs, discussion sites, and sharing economy networks.

In some embodiments, the memory 600 may be a storage device/storage system, including direct-attached storage (direct-attached storage), a network-attached storage (network-attached storage), and a storage area network (storage area network). The storage device includes, but is not limited to, various common storage devices, such as solid-state storage devices (solid-state disk, solid-state hybrid disk, etc.), mechanical hard disk, USB flash memory, memory stick, memory card (e.g., CF, SD, etc.), other drives (e.g., CD, DVD, HD DVD, Blu-ray, etc.), Random Access Memory (RAM), and Read Only Memory (ROM). The RAM includes but is not limited to: decimal count tubes, delay line memories, Williams tubes, Dynamic Random-Access Memories (DRAMs), Static Random-Access Memories (SRAMs), thyristor random access memories (T-RAMs), zero-capacitance random access memories (Z-RAMs), and the like. In some embodiment, ROM comprises, but is not limited to: bubble memory, magnetic button wire memory, thin film memory, magnetic plated wire memory, magnetic core memory, magnetic drum memory, optical disk drive, hard disk, magnetic tape, early NVRAM (non-volatile memory), phase change memory, magneto resistive random access memory, ferroelectric random access memory, nonvolatile SRAM, flash memory, EEPROM, erasable programmable read-only memory, shielded read-stack memory, floating gate random access memory, nano-RAM, racetrack memory, variable resistive memory, and programmable metallization cells, etc.

It should be understood that the above-described storage device/storage system is exemplary and any other storage device/storage system can be adopted in various embodiments of this disclosure.

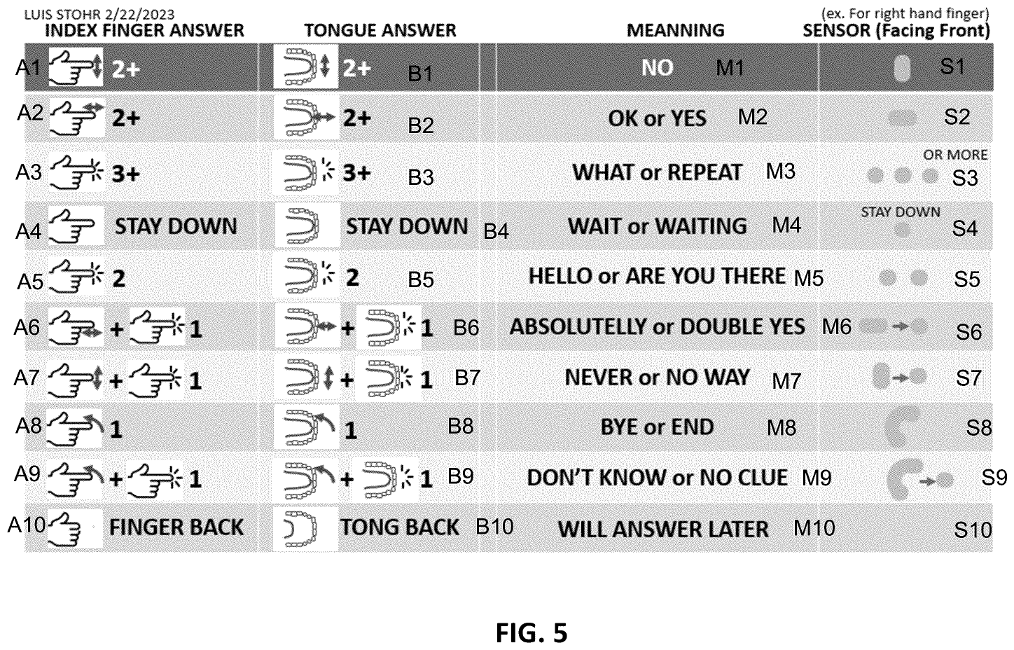

FIG. 5 generally depicts the predetermined pattern of the motions of the human body, including the human finger and the human tongue according to an aspect of the embodiment.

Referring to FIG. 5, the predetermined pattern of the motions of the human body (human fingers) may comprise index finger answer A1-A10, wherein the processor executes the instructions to: if the pattern of the motions of the human body (human fingers) matches with the predetermined pattern of motions of the human body (human fingers), including the index finger answer A1-A10, the processor may generate responding data, including meaning patterns M1-M10 based on the predetermined pattern of motions of the human body (human fingers), including the index finger answer A1-A10.

In some embodiments, the accelerometer 101 of the wearable device 100 (as shown in FIG. 1) may comprise a sensor to detect the pattern of the motions of the human body (human fingers), including sensor patterns S1-S10.

Continuing to FIG. 5, the predetermined pattern of the motions of the human body (human tongue) may comprise tongue answer B1-B10, wherein the processor executes the instructions to: if the pattern of the motions of the human tongue matches with the predetermined pattern of the motions of the human tongue, including the tongue answer B1-B10, the processor may generate the first responding data, including meaning patterns M1-M10 based on the predetermined pattern of motions of the human tongue, including the tongue answer B1-B10.

In some embodiments, the switch sensor 201 of the tongue sensor device 200 (as shown in FIG. 4) may detect the pattern of the motions of the human tongue, including sensor patterns S1-S10.

In some embodiments, the predetermined pattern of motions of the human body (human fingers and/or human tongue) comprises a direction, a frequency, and a dwell time of the motions of the human body (human fingers and/or human tongue).

It should be understood that the above-described pattern and predetermined pattern of motions of the human body (human fingers and/or human tongue) are exemplary and any other pattern and predetermined pattern of motions of the human body (human fingers and/or human tongue) can be adopted in various embodiments of this disclosure.

In some embodiments, the responding data and/or the first responding data comprises readable word information, audio information, and/or vibration information.

It should be noted that, in some embodiments, the human body (human fingers and/or human tongue) is exemplary, and any other life/being, such as dogs, cats, or pets, can be adopted in various embodiments of this disclosure.

In some embodiments, the wearable device 100 and/or the tongue sensor device 200 may include the use of metal cables, optical cables, or a hybrid of metal and optical cables, such as: coaxial cables, communication cables, flexible cables, spiral cables, non-metallic sheathed cables, multi-core cables, twisted pair cables, ribbon cables, shielded cables, telecommunication cables, twin cables, parallel twin wires, and twisted pairs. The above-described examples are merely for the convenience of illustration, and the medium for wired connection may be other types of transmission medium, such as other transmission mediums of electrical or optical signals. Wireless connections include, but are not limited to, radio communications, free-space optical communications, acoustic communications, electromagnetic induction, and the like. Wherein the radio communication includes, but is not limited to, IEEE802.11 series of standards, IEEE802.15 series of standards (e.g., Bluetooth and ZigBee technologies, etc.), first generation mobile communication technologies, second generation mobile communication technologies (e.g., FDMA, TDMA, SDMA, CDMA, and SSMA, etc.), general packet radio service technologies, third generation mobile communication technologies (e.g., CDMA2000, WCDMA, TD-SCDMA, and WiMAX, etc.), fourth generation mobile communication technologies (e.g., TD-LTE and FDD-LTE, etc.), satellite communication (e.g., GPS technologies, etc.), Near Field Communication (NFC), and other technologies operating in ISM band (e.g., 2.4 GHz, etc.); free space optical communications include, but are not limited to, visible light, infrared signals, and the like; acoustic communications include, but are not limited to, acoustic waves, ultrasonic signals, and the like; electromagnetic induction includes, but is not limited to, near field communication techniques and the like. The above examples are for the convenience of illustration only, and the medium for the wireless connection may be of other types, such as Z-wave technology, other premium civilian radio bands, and military radio bands, among others.

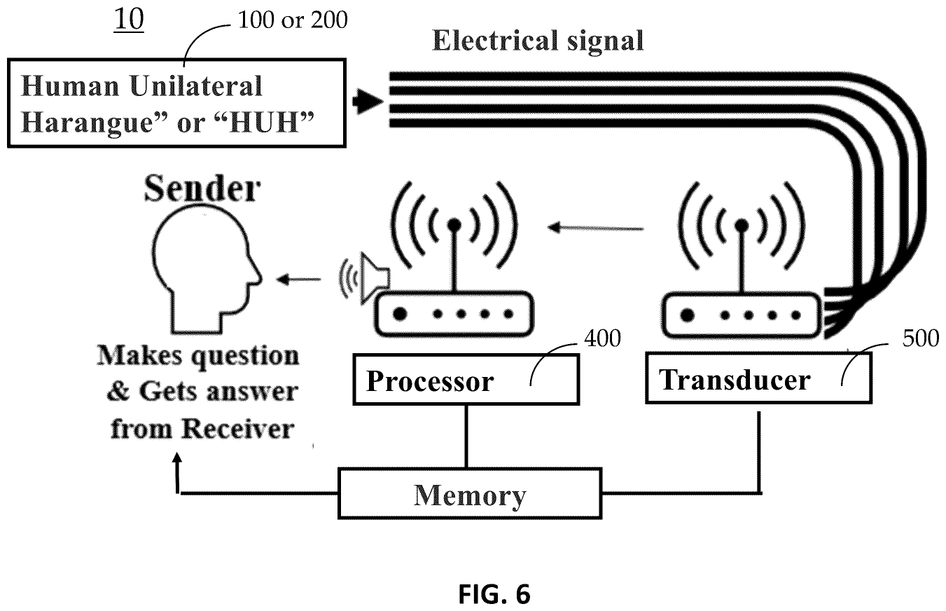

FIG. 6 generally depicts the communication system 10 through the human body according to an aspect of the embodiment.

Referring to FIG. 6, the system 10 may further comprise a transducer 500 to obtain the electrical signal/the first electrical signal from the wearable device 100 and/or the tongue sensor device 200 or directly obtain the responding data and/or the first responding data. In some embodiments, the electrical signal and/or the first electrical signal may be transmitted to the processor 400, wherein the processor 400 may transduce the electrical signal and/or the first electrical signal to the responding data and/or the first responding data, and in such a manner, the sender may receive the responding data and/or the first responding data.

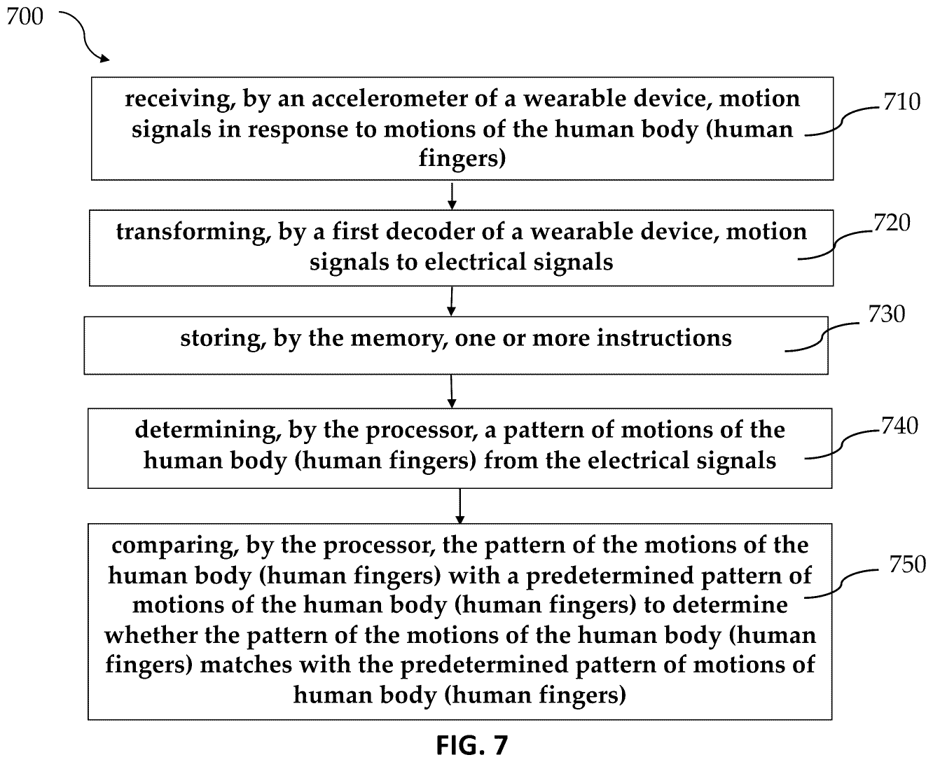

FIGS. 7-10 generally depict a communication method 700 through a human body by a communication system according to an aspect of the embodiment.

Referring to FIG. 7, the method 700 may comprise:
receiving, by an accelerometer of a wearable device, motion signals in response to motions of the human body (human fingers) 710;
transforming, by a first decoder of a wearable device, motion signals to electrical signals 720;
storing, by the memory, one or more instructions 730;
determining, by the processor, a pattern of the motions of the human body (human fingers) from the electrical signals 740; and
comparing, by the processor, the pattern of the motions of the human body (human fingers) with a predetermined pattern of motions of the human body (human fingers) to determine whether the pattern of the motions of the human body (human fingers) matches with the predetermined pattern of the motions of the human body (human fingers) 750.

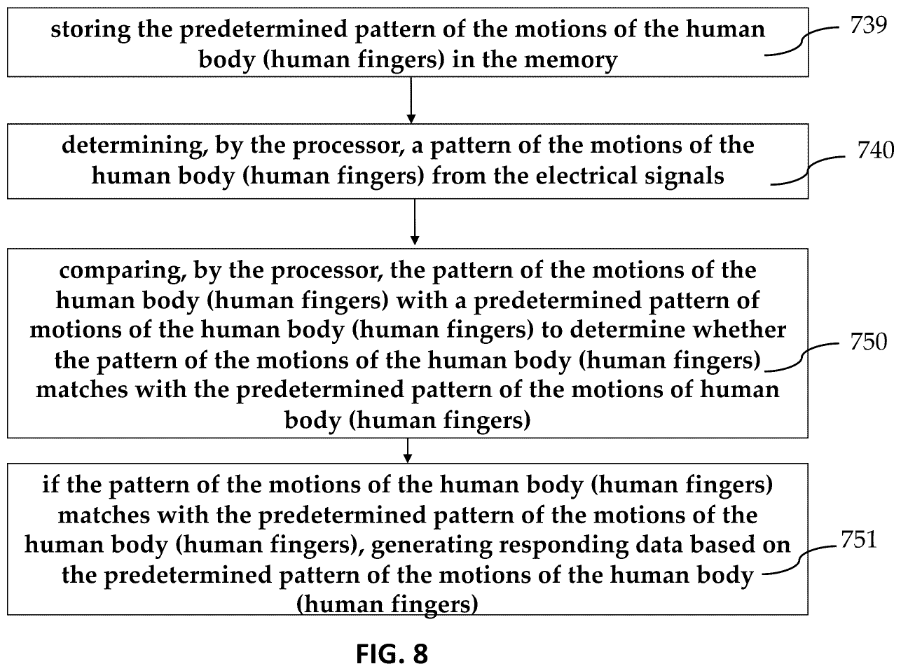

Referring to FIG. 8, the step of comparing, by the processor, the pattern of the motions of the human body (human fingers) with a predetermined pattern of the motions of the human body (human fingers) to determine whether the pattern of the motions of the human body (human fingers) matches with the predetermined pattern of the motions of the human body (human fingers) 750 may further comprise steps of:
if the pattern of the motions of the human body (human fingers) matches with the predetermined pattern of motions of the human body (human fingers), generating responding data based on the predetermined pattern of motions of the human body (human fingers) 751.

In some embodiment, the step of determining, by the processor, the pattern of the motions of the human body (human fingers) from the electrical signals 740 may further comprise steps of: storing the predetermined pattern of motions of the human body (human fingers) in the memory 739.

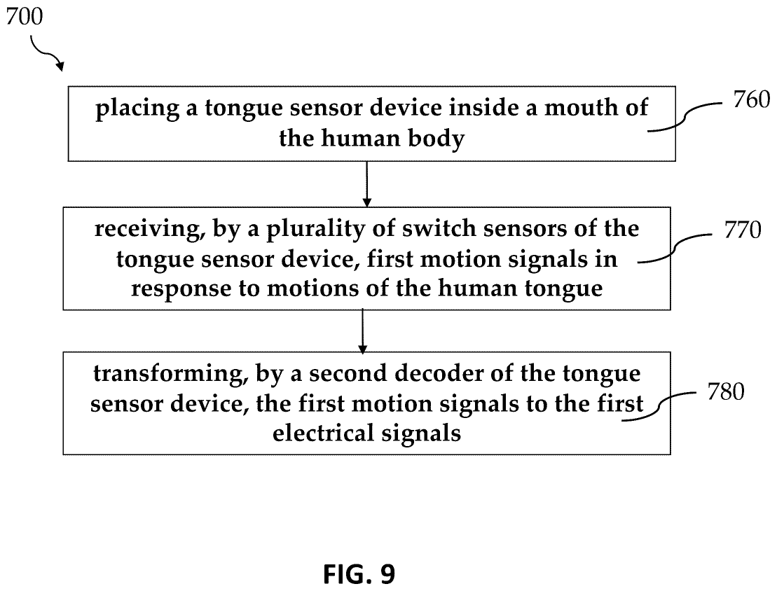

Referring to FIG. 9, the method 700 may further comprise steps of:
placing a tongue sensor device inside a mouth of the human body 760;
receiving, by a plurality of switch sensors of the tongue sensor device, first motion signals in response to motions of human tongue 770; and
transforming, by a second decoder of the tongue sensor device, the first motion signals to the first electrical signals 780.

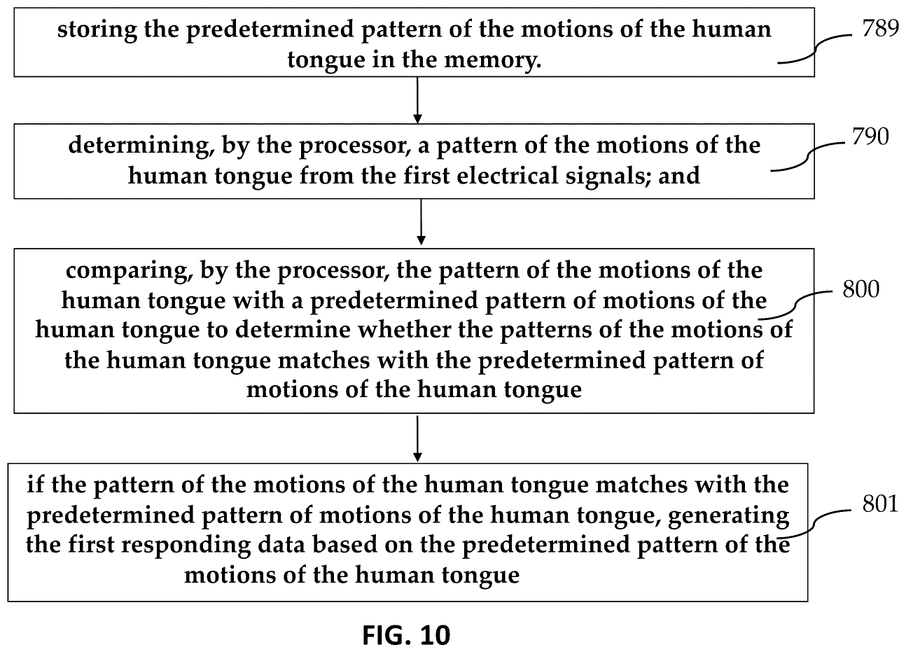

In some embodiments, referring to FIG. 10, the method 700 may further comprise steps of:
determining, by the processor, a pattern of the motions of the human tongue from the first electrical signals 790; and
comparing, by the processor, the pattern of the motions of the human tongue with a predetermined pattern of motions of the human tongue to determine whether the pattern of the motions of the human tongue matches the predetermined pattern of the motions of the human tongue 800.

In some embodiments, the step of comparing the pattern of the motions of the human tongue with a predetermined pattern of motions of the human tongue to determine whether the pattern of the motions of the human tongue matches the predetermined pattern of motions of the human tongue may further comprise steps of:
if the pattern of the motions of the human tongue matches with the predetermined pattern of motions of the human tongue, generating first responding data, including readable word information, audio information and/or vibration information, based on the predetermined pattern of the motions of the human tongue 801.

In some embodiments, the step of determining, by the processor, the pattern of the motions of the human tongue from the first electrical signals may further comprise steps of:
storing the predetermined pattern of the motions of the human tongue in the memory 789.

Similarly, while operations and/or methods may be depicted in the drawings in a particular order, this should not be understood as requiring that such operations be performed in the particular order shown or in sequential order, or that all illustrated operations and/or methods steps be performed, to achieve desirable results. In certain circumstances, multitasking and parallel processing may be advantageous.

### Detailed Descriptions of the Experimentation Approach

The following experiment aims to prove that responding using the HUH (the wearable device and/or the tongue sensor device) method and apparatus offers an answering approach with the lowest distraction possible. In the following experiments, the sender verbally asks five questions to the receiver who answers these questions using HUH (the wearable device and/or the tongue sensor device), while performing a series of ten simple addition mathematical equations and writing its solution on a paper. This activity is repeated twice, “1” responding verbally and “2” responding with extremely simple body gestures instead of using the HUH (the wearable device and/or the tongue sensor device). The receiver has to perform the equations and answer the verbal questions as soon as possible. Times spent by the receiver writing the answers to the ten equations and answering the verbal questions are recorded and later approaches “1” and “2,” which are compared with the reference HUH (the wearable device and/or the tongue sensor device). The timing will quantify the efficiency while writing the solutions of the equations while being distracted with the verbal questions, and the results are compared to find which method in which the receiver is exposed is faster and more efficient under multitasking conditions.

Before the experimentation, the receivers (under the experimentation analysis) have fifteen minutes to practice using the HUH (the wearable device and/or the tongue sensor device) method and apparatus, and fifteen minutes. At the same time, the experimentation will be explained to the receiver. The parameters such as mounting the mouth bracket, volume, and locations, are adjusted for the receiver to be comfortable. The experiment is repeated if any of the verbal questions or the equation's written answers are answered wrongly. If the receiver has complications responding to the questions (gets stocked), the experimentation is repeated.

For this experimentation, five persons are chosen with different ages to perform the experimentation—persons who are tested at the ages of 11, 35, 44, 55, and 82. The locations used to experiment are different, but the receivers are under any additional distraction. To perform this experimentation, the HUH (the wearable device and/or the tongue sensor device) device prototype is built and used by a small group of people of a wide range of ages. The experimentation utilized a series of simple questions (or mathematical equations) asked by the sender and responded to by the receiver. There are various levels of complexity for questions and equations, and some might be difficult for certain people under the tests; this is why the questions, mathematical equations, and the same questions and equations for each person are tested under experimentation.

The HUH (the tongue sensor device) apparatus with a proper mounting which is developed on anti-allergic latex, mounting adequate switches (waterproof, size as the tongue's tip or less than 10 mm, low activation force), is placed on the area behind the lower mandibular area, and the first electrical signal from the switch sensors are transmitted to the transducer located outside the mouth. Since this experimentation is not proven for mass production or long-term use, which depends on the weight, safety, and size of the transmitting section of the HUH (the tongue sensor device), the transducer may not be located on the mouth mount.

Referring back to FIG. 6, the electrical signals/the first electrical signal may be transduced to RF responding signals which contain the answer information and transformed them the processor through the media, wherein the RF responding signals may be transformed to the responding data/the first responding data by the processor. In such a manner, the responding data may be perceived and understood by a second person or the sender who verbally is performing the pre-assigned and written questions. The second person or the sender who asks the questions will record the answers from the receiver through an external comeback device.

### Documents Used in Experimentation

There are two forms used on experimentation:

The first form: five different paper sheets are used as a task for the receiver of the verbal questions to write down the solutions of these math equations simultaneously when they are answering the simple verbal questions from the receiver. The paper sheets have simple adding equations such as: “6+3=, 2+5=, 7+1=, 2+4=, 7+2=, 5+4=, 1+2=, 6+3=, 2+6=, 4+4=, 1+1=, 3+5=, 3+3=, 1+8=, 2+2=”. Receivers may answer the equations, and the answers may be recorded as “Equations with no Questions.”

The second form: The sender uses another paper sheet with the three questions to ask the receiver while the receivers write the solutions for the equations from the given paper sheet. The sender records each response time spent by the receiver as “Response time.” The three questions are: “Are you sitting down?”, “Is it raining now?” and “Did you eat breakfast today.” The receivers are directedly responding as soon as possible.

### Experimentation Results

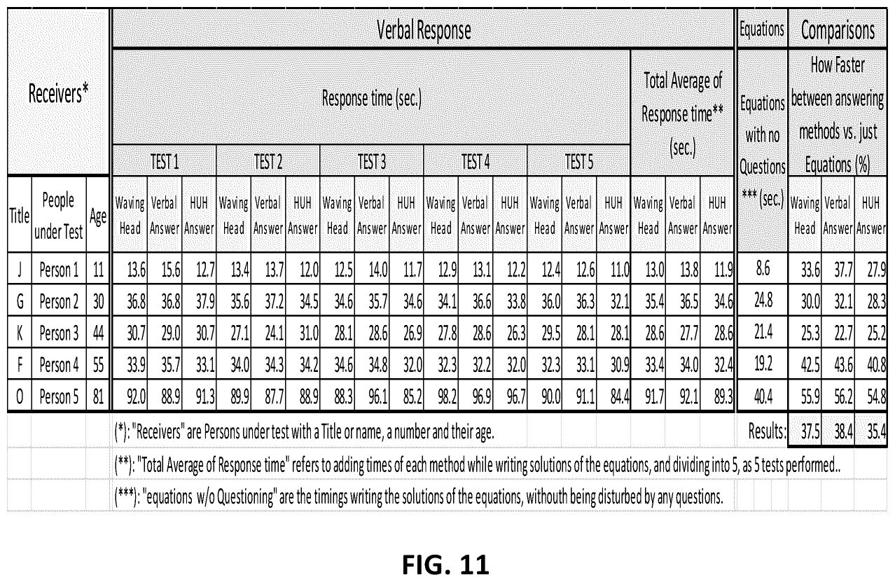

The results during experimentation are shown in FIG. 11.

Testing conditions: The sender who is asking the questions and recording the timings is Luis Stohr. Tests are performed under silent conditions. Only two people are present and sitting at a table during the tests, such as the sender and the receiver, who are tested under the analysis of the experimentation. Time is recorded using mobile phones' timers.

### Analysis of Experimentation Results

1. Based on the Table results in FIG. 11, the fastest performance between responses of people under tests is using the HUH with 34.4% more time to solve the equations, which is our test value to define distraction, with respect to the 38.4% verbal answers and the 37.5% waving head gestures answers.
2. Five tests with five different but simple adding equations sheets are used for average performance and to avoid testing errors.
3. People under the test commented that the HUH answering method is the most seamless and faster way to answer, although the device in the buccal cavity is a little uncomfortable.
4. Based on the Table results in FIG. 11, there is a large variation in times to write the solutions of the equations and the people's age, but this is not part of the purpose of the tests.

### Experimentation Results

The method and apparatus presented in this invention offer a new efficient communication approach where the sender who asks questions can reach the receiver, wherein the receiver may answer the questions efficiently, with less body energy consumption and lower distraction. Today and in the future, people will perform several duties, and communication between people will increase rapidly.

Many alterations and modifications may be made by those having ordinary skill in the art without departing from the spirit and scope of the disclosed embodiments. Therefore, it must be understood that the illustrated embodiments have been set forth only. For example, it should not be taken as limiting the embodiments as defined by the following claims. For example, even though the elements of a claim are set forth below in a certain combination, it must be expressly understood that the embodiment includes other combinations of fewer, more, or different elements, which are disclosed herein even when not initially claimed in such combinations.

Thus, specific embodiments and applications of the communication method and system using the human body have been disclosed. It should be apparent, however, to those skilled in the art that many more modifications besides those already described are possible without departing from the disclosed concepts herein. Therefore, the disclosed embodiments are not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced. Insubstantial changes from the claimed subject matter as viewed by a person with ordinary skill in the art, now known or later devised, are expressly contemplated as equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be substituted, and what essentially incorporates the essential idea of the embodiments. In addition, where the specification and claims refer to at least one of something selected from the group consisting of A, B, C . . . and N, the text should be interpreted as requiring at least one element from the group which includes N, not A plus N, or B plus N, etc.

The words used in this specification to describe the various embodiments are to be understood not only in the sense of their commonly defined meanings but to include by special definition in this specification structure, material, or acts beyond the scope of the commonly defined meanings. Thus, if an element can be understood in this specification as including more than one meaning, its use in a claim must be understood as being generic to all possible meanings supported by the specification and the word itself.

Therefore, the definitions of the words or elements of the following claims include not only the combination of elements set forth but all equivalent structures, material, or acts for performing substantially the same function in the same way to obtain the same result. Therefore, it is contemplated that an equivalent substitution of two or more elements may be made for any of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

1. A communication system through a human body, comprising:
a wearable device comprising an accelerometer configured to receive motion signals in response to motions of the human body and a first decoder configured to transform motion signals into electrical signals;
a memory configured to store one or more instructions; and
a processor communicated with the wearable device and configured to execute the instructions to:
determine a pattern of motions of the human body from the electrical signals; and
compare the pattern of the motions of the human body with a predetermined pattern of motions of the human body to determine whether the pattern of the motions of the human body matches with the predetermined pattern of the motions of human body,
wherein the wearable device is worn on fingers of the human body to detect motions of the human fingers, or a tongue sensor device placed inside a mouth of the human body and communicated with the processor,
wherein the processor executes the instructions that if the pattern of the motions of the human body matches with the predetermined pattern of motions of the human body including either human finger or tongue, generating responding data based on the predetermined pattern of motions of the human body; and the responding data comprises human-understandable wording information, audio information and/or vibration information.

2. (canceled)

3. (canceled)

4. The communication system of claim 1, the instructions for determining the pattern of the motions of the human body from the electrical signals comprises instructions for the system to:
store the predetermined pattern of the motions of the human body in the memory.

5. The communication system of claim 1, wherein the predetermined pattern of motions of the human body comprises a direction, a frequency, and a dwell time of the motions of the human body.

6. (canceled)

7. The communication system of claim 1, wherein the tongue sensor device comprises a plurality of switch sensors to receive first motion signals in response to motions of a human tongue.

8. The communication system of claim 7, wherein the tongue sensor device comprises a second decoder configured to transform first motion signals to first electrical signals.

9. The communication system of claim 8, wherein the processor executes the instructions to:
determine a pattern of the motions of the human tongue from the first electrical signals; and
compare the pattern of the motions of the human tongue with a predetermined pattern of motions of the human tongue to determine whether the pattern of the motions of the human tongue matches the predetermined pattern of the motions of the human tongue.

10. (canceled)

11. The communication system of claim 9, the instructions for determining the pattern of the motions of the human tongue from the first electrical signals comprises instructions for the system to:
store the predetermined pattern of the motions of the human tongue in the memory.

12. (canceled)

13. A communication method through a human body, comprising:
receiving, by an accelerometer of a wearable device, motion signals in response to motions of the human body;
transforming, by a first decoder of a wearable device, motion signals to electrical signals;
storing, by the memory, one or more instructions;
determining, by the processor, a pattern of motions of the human body from the electrical signals; and
comparing, by the processor, the pattern of the motions of the human body with a predetermined pattern of motions of the human body to determine whether the pattern of the motions of the human body matches the predetermined pattern of motions of human body,
wherein the wearable device is worn on fingers of the human body to detect motions of the human fingers, or a tongue sensor device placed inside a mouth of the human body and communicated with the processor,
wherein comparing, by the processor, the pattern of the motions of the human body with a predetermined pattern of motions of the human body to determine whether the pattern of the motions of the human body matches with the predetermined pattern of motions of human body further comprises:
if the pattern of the motions of the human body matches with the predetermined pattern of motions of the human body, generating responding data based on the predetermined pattern of motions of the human body; and the responding data comprises human-understandable wording information, audio information and/or vibration information.

14. (canceled)

15. (canceled)

16. The communication method of claim 13, wherein determining, by the processor, the pattern of the motions of the human body from the electrical signals further comprises: storing the predetermined pattern of motions of the human body in the memory.

17. The communication method of claim 13, further comprising steps of:
receiving, by a plurality of switch sensors of the tongue sensor device, first motion signal in response to motions of human tongue; and
transforming, by a second decoder of the tongue sensor device, the first motions signals to first electrical signals.

18. The communication method of claim 17, further comprising steps of:
determining, by the processor, a pattern of the motions of the human tongue from the first electrical signals; and
comparing, by the processor, the pattern of the motions of the human tongue with a predetermined pattern of motions of the human tongue to determine whether the pattern of the motions of the human tongue matches the predetermined pattern of motions of the human tongue.

19. (canceled)

20. The communication method of claim 18, wherein determining, by the processor, the pattern of the motions of the human tongue from the first electrical signals, further comprises steps of:
storing the predetermined pattern of the motions of the human tongue in the memory.
